(19) United States
(12) Patent Application Publication
(10) Pub. No.: US 2024/0262775 A1
(43) Pub. Date: Aug. 8, 2024

(54) Methods to synthesize cyclopentenes and cyclopentadienes by ring contraction

(71) Applicant: Natural Extraction Systems, LLC, Boulder, CO (US)

(72) Inventor: C. Russell Thomas, Boulder, CO (US)

(21) Appl. No.: 18/560,625

(22) PCT Filed: May 10, 2022
(86) PCT No.: PCT/US2022/028616

§ 371 Date: Nov. 13, 2023

Related U.S. Application Data
(60) Provisional application No. 63/187,216, filed on May 11, 2021.

(51) Int. Cl. C07C 45/67

(52) U.S. Cl. CPC C07C 45/67

(57) ABSTRACT

Various aspects of this disclosure relate to (1) methods to perform a ring contraction in the gas phase, (2) compositions obtainable by such methods, and (3) reaction vessels that contain compositions that comprise either a reactant or a product of the ring contraction.

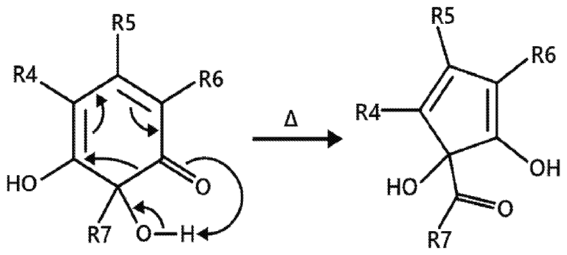

### CROSS-REFERENCE TO RELATED APPLICATIONS

This International Application claims priority to U.S. Provisional Patent Application No. 63/187,216, filed May 11, 2021, which is incorporated by reference in its entirety.

### BACKGROUND

Synthetic routes to cyclopentadienes are limited, for example, because cyclopentadienes rapidly undergo sigmatropic rearrangements and Diels-Alder reactions. Hops produces the cyclohexadiene humulone that can be converted into a cyclopentadiene in protic polar solvents. The cyclopentadiene product is unstable in protic polar solvents, however, and converts into a keto tautomer isohumulone. A method to manufacture isohumulone or its enol tautomer without solvation in a protic polar solvent would allow for differentiated hops products, for example, to allow the addition of isohumulone into beer from an organic hops extract or during a dry-hopping process. Generally applicable methods to produce cyclopentadienes in the absence of protic polar solvent would allow new organic synthesis strategies.

### SUMMARY

Various aspects of this disclosure relate to the discovery that the dry distillation of volatiles from Humulus lupulus resulted in the production of bitter acids. These bitter acids were previously believed to require a protic polar solvent to manufacture. Without limiting this disclosure or any patent claim that matures from this document, either (1) Humulus lupus may comprise proton donors and/or proton acceptors in sufficient proximity to alpha acids to catalyze their conversion into iso-alpha acids in the absence of solvation by a polar protic solvent, or (2) alpha acids may be capable of conversion into iso-alpha acids through an intramolecular reaction mechanism, for example, that comprises the ring contraction depicted in the FIGURE. Regardless of the reaction mechanism, the conversion of alpha acids into iso-alpha acids in the absence of solvation by a polar protic solvent provides a previously unappreciated opportunity to isolate the cyclopentadiene intermediates in the conversion of alpha acids to iso-alpha acids and to isolate other cyclopentadienes produced from different reactants. Regardless of the reaction mechanism, the conversion of alpha acids into iso-alpha acids in the absence of solvation by a polar protic solvent provides a new strategy to produce cyclopentenes. Without limiting the disclosure or any patent claim that matures from this document, a new strategy to synthesize cyclopentenes would likely favor a novel stereochemical fingerprint. The novel strategy allows the production of differentiated hops products such as hops extract that comprises iso-alpha acids or such as a minimally-processed hops that comprises iso-alpha acids. The novel strategy similarly allows a new synthetic route to cyclopentenes.

### BRIEF DESCRIPTION OF THE DRAWING

The FIGURE depicts an intramolecular reaction to convert a cyclohexadiene into a cyclopentadiene, and the FIGURE does not limit this disclosure or any patent claim that matures from this document.

### DETAILED DESCRIPTION

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentadiene of a product while the condensed phase is suspended in the gas phase, wherein: the cyclohexadiene comprises a first carbon atom, a second carbon atom, a third carbon atom, a fourth carbon atom, a fifth carbon atom, and a sixth carbon atom, wherein the first carbon atom is bound to the second carbon atom by a single bond, the second carbon atom is bound to the third carbon atom by a single bond, the third carbon atom is bound to the fourth carbon atom by a double bond, the fourth carbon atom is bound to the fifth carbon atom by a single bond, the fifth carbon atom is bound to the sixth carbon atom by a double bound, and the sixth carbon atom is bound to the first carbon atom by a single bond; the cyclopentadiene comprises the first carbon atom, the third carbon atom, the fourth carbon atom, the fifth carbon atom, and the sixth carbon atom, wherein the first carbon atom is bound to the third carbon atom by a single bond, the third carbon atom is bound to the fourth carbon atom by a single bond, the fourth carbon atom is bound to the fifth carbon atom by a double bond, the fifth carbon atom is bound to the sixth carbon atom by a single bound, and the sixth carbon atom is bound to the first carbon atom by a double bond; the product is a structural isomer of the reactant; the product comprises the second carbon atom, which is bound to the third carbon atom of the cyclopentadiene by a single bond; the first carbon atom of the cyclohexadiene is an unsaturated carbon atom that is substituted with an oxo, thioxo, or imino substituent, and the first carbon atom of the cyclopentadiene is an unsaturated carbon atom that is substituted with a hydroxy, sulfanyl, or amino substituent, respectively; the second carbon atom of the cyclohexadiene is a saturated carbon atom that is substituted with a hydroxy, sulfanyl, or amino substituent, and the second carbon atom of the product is an unsaturated carbon atom that is substituted with an oxo, thioxo, or imino substituent, respectively; and the third carbon atom of the cyclohexadiene is an unsaturated carbon atom that is substituted with a hydroxy, sulfanyl, or amino substituent, and the third carbon atom of the cyclopentadiene is a saturated carbon atom that is substituted with a hydroxy, sulfanyl, or amino substituent, respectively.

“Comprising” and “comprise” refer to open sets such that a method can comprise additional, undisclosed steps.

In some embodiments, the reactant has General Structure Ia; the product has General Structure IIa; either (i) R1a is oxo, and R1b is hydroxy, (ii) R1a is imino, and Rib is amino, or (iii) R1a is thioxo, and R1b is sulfanyl; either (i) R2a is hydroxy, and R2b is oxo, (ii) R2a is amino, and R2b is imino, or (iii) R2a is sulfanyl, and R2b is thioxo; R3 is either hydroxy, amino, or sulfanyl; R4, R5, R6, and R7 are each independently selected from hydro, hydroxy, amino, cyano, halo, and a branched-or-unbranched, saturated-or-unsaturated, C1-C11 hydrocarbon group that is optionally substituted with one or more of hydroxy, oxo, sulfanyl, thioxo, thio, amino, cyano, isocyano, halo, phenyl, oxa, and a saturated-or-unsaturated cycloalkyl that is optionally substituted with one or more of hydroxy, methoxy, oxo, formyl, acetyl, methyl, ethyl, 2-propyl, and propen-2-yl; and the dotted line in General Structure IIa depicts a double bond.

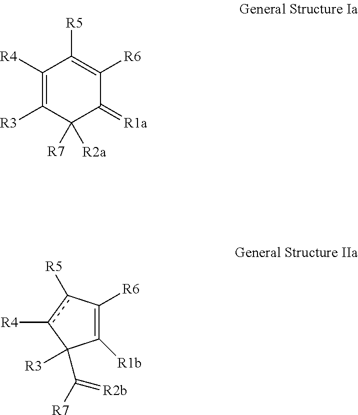

“Halo” is selected from fluoro, chloro, bromo, and iodo.

“Branched-or-unbranched, saturated-or-unsaturated, C1-C11 hydrocarbon group” refers to a branched-or-unbranched, saturated-or-unsaturated hydrocarbon chain that comprises exactly 1, 2, 3, 4, 5, 6, 7, 8, 9, 10, or 11 carbon atoms.

“Substituted with one or more of hydroxy, oxo, sulfanyl, thioxo, thio, amino, cyano, isocyano, halo, phenyl” refers to the substitution of one or more protons of a hydrocarbon chain with one or more of hydroxy, oxo, sulfanyl, thioxo, thio, amino, cyano, isocyano, halo, or phenyl, respectively; when a branched-or-unbranched, saturated-or-unsaturated, C1-C11 hydrocarbon group is substituted with oxo, then two protons of the same carbon atom of the branched-or-unbranched, saturated-or-unsaturated, C1-C11 hydrocarbon group are substituted with the oxo such that the substituted carbon atom is an unsaturated carbon atom because of the substitution with oxo. The carbon atoms of cyano, isocyano, and phenyl are included when counting the number of carbon atoms in a branched-or-unbranched, saturated-or-unsaturated, C1-C11 hydrocarbon group.

“Substituted with . . . oxa” refers to the substitution of a carbon atom of a hydrocarbon chain and two protons that are covalently bound to the carbon atom of the hydrocarbon chain with an oxygen atom.

“Substituted with . . . a saturated-or-unsaturated cycloalkyl” refers to the substitution of one or two protons of a hydrocarbon chain with a saturated-or-unsaturated carbon homocycle; when one proton of the hydrocarbon chain is substituted with the saturated-or-unsaturated carbon homocycle, then the saturated-or-unsaturated carbon homocycle does not include any carbon atom of the hydrocarbon chain; and when two protons of the hydrocarbon chain are substituted with the saturated-or-unsaturated carbon homocycle, then the saturated-or-unsaturated carbon homocycle includes one or more carbon atoms of the hydrocarbon chain. The carbon atoms of the saturated-or-unsaturated carbon homocycle are included when counting the number of carbon atoms in a branched-or-unbranched, saturated-or-unsaturated, C1-C11 hydrocarbon group.

“Substituted with one or more of hydroxy, methoxy, oxo, formyl, acetyl, methyl, ethyl, 2-propyl, and propen-2-yl” refers to the substitution of one or more protons of a saturated-or-unsaturated cycloalkyl with one or more of hydroxy, methoxy, oxo, formyl, acetyl, methyl, ethyl, 2-propyl, and propen-2-yl, respectively; when a saturated-or-unsaturated cycloalkyl is substituted with oxo, then two protons of the same carbon atom of the saturated-or-unsaturated cycloalkyl are substituted with the oxo such that the substituted carbon atom is an unsaturated carbon atom because of the substitution with oxo.

In some embodiments, R1a and R2b are each oxo; and R1b, R2a, and R3 are each hydroxy.

In some embodiments, one of R4, R5, and R6 is hydro, hydroxy, methoxy, methyl, or formyl; the other two of R4, R5, and R6 are each independently selected from hydro, hydroxy, methoxy, formyl, acetyl, 1-oxopropyl, 1-oxobutyl, 2-methyl-1-oxopropyl, 2-methyl-1-oxobutyl, 3-methyl-1-oxobutyl, 4-methyl-1-oxopentyl, methyl, ethyl, vinyl, ethynyl, propyl, prop-2-yl, prop-1-enyl, prop-2-enyl, propen-2-yl, prenyl, and geranyl; and R7 is methyl, ethyl, vinyl, propyl, prop-2-yl, prop-1-enyl, prop-2-enyl, propen-2-yl, prenyl, or geranyl.

In some embodiments, R1a and R2b are each oxo; R1b, R2a, R3, and R5 are each hydroxy; R4 and R7 are each prenyl; and R6 is 1-oxopropyl, 2-methyl-1-oxopropyl, 2-methyl-1-oxobutyl, 3-methyl-1-oxobutyl, or 4-methyl-1-oxopentyl.

In some embodiments, R6 is 1-oxopropyl.

In some embodiments, R6 is 2-methyl-1-oxopropyl.

In some embodiments, R6 is 2-methyl-1-oxobutyl.

In some embodiments, R6 is 3-methyl-1-oxobutyl.

In some embodiments, R6 is 4-methyl-1-oxopentyl.

In some embodiments, the method comprises converting the product into a tautomer, wherein: the product and the tautomer are structural isomers; the product has General Structure IIa, wherein R5 is hydroxy, and the dotted line in General Structure IIa depicts a double bond; and the tautomer has General Structure IIa, wherein R5 is oxo, and the dotted line in General Structure IIa depicts a single bond. When R5 is oxo, then the single line that depicts a bond between R5 and the carbon homocycle of General Structure IIa depicts a double bond.

In some embodiments, R3 of the product comprises a proton; and converting the product into the tautomer comprises transferring the proton of R3 of the product to the fourth carbon atom of the cyclopentadiene of the product.

In some embodiments, R1b of the product comprises a proton; and converting the product into the tautomer comprises transferring the proton of R1b of the product to R3 of the product.

In some embodiments, R5 of the product comprises a proton; R6 of the product comprises a carbonyl oxygen; and converting the product into the tautomer comprises transferring the proton of R5 of the product to the carbonyl oxygen of R6 of the product.

In some embodiments, the reactant is 3,5,6-trihydroxy-2-(3-methyl-1-oxobutyl)-4,6-diprenylcyclohexa-2,4-dien-1-one; the product is 2-(3-methyl-1-oxobutyl)-5-(4-methyl-1-oxopent-3-enyl)-4-prenylcyclopenta-1,3-diene-1,3,5-triol; and the tautomer is 3,4-dihydroxy-2-(3-methyl-1-oxobutyl)-4-(4-methyl-1-oxopent-3-enyl)-5-prenylcyclopent-2-en-1-one.

In some embodiments, the reactant is 3,5,6-trihydroxy-2-(2-methyl-1-oxobutyl)-4,6-diprenylcyclohexa-2,4-dien-1-one; the product is 2-(2-methyl-1-oxobutyl)-5-(4-methyl-1-oxopent-3-enyl)-4-prenylcyclopenta-1,3-diene-1,3,5-triol; and the tautomer is 3,4-dihydroxy-2-(2-methyl-1-oxobutyl)-4-(4-methyl-1-oxopent-3-enyl)-5-prenylcyclopent-2-en-1-one.

In some embodiments, the reactant is 3,5,6-trihydroxy-2-(2-methyl-1-oxopropyl)-4,6-diprenylcyclohexa-2,4-dien-1-one; the product is 2-(2-methyl-1-oxopropyl)-5-(4-methyl-1-oxopent-3-enyl)-4-prenylcyclopenta-1,3-diene-1,3,5-triol; and the tautomer is 3,4-dihydroxy-2-(2-methyl-1-oxopropyl)-4-(4-methyl-1-oxopent-3-enyl)-5-prenylcyclopent-2-en-1-one.

In some embodiments, the reactant is 3,5,6-trihydroxy-2-(1-oxopropyl)-4,6-diprenylcyclohexa-2,4-dien-1-one; the product is 2-(1-oxopropyl)-5-(4-methyl-1-oxopent-3-enyl)-4-prenylcyclopenta-1,3-diene-1,3,5-triol; and the tautomer is 3,4-dihydroxy-2-(1-oxopropyl)-4-(4-methyl-1-oxopent-3-enyl)-5-prenylcyclopent-2-en-1-one.

In some embodiments, the reactant is 3,5,6-trihydroxy-2-(4-methyl-1-oxopentyl)-4,6-diprenylcyclohexa-2,4-dien-1-one; the product is 2-(4-methyl-1-oxopentyl)-5-(4-methyl-1-oxopent-3-enyl)-4-prenylcyclopenta-1,3-diene-1,3,5-triol; and the tautomer is 3,4-dihydroxy-2-(4-methyl-1-oxopentyl)-4-(4-methyl-1-oxopent-3-enyl)-5-prenylcyclopent-2-en-1-one.

In some embodiments, R4 of the product is not hydro; and converting the product into the tautomer is stereoselective.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentadiene of a product while the condensed phase is suspended in the gas phase, wherein: the reactant is 3,5,6-trihydroxy-2-(3-methyl-1-oxobutyl)-4,6-diprenylcyclohexa-2,4-dien-1-one; and the product is 2-(3-methyl-1-oxobutyl)-5-(4-methyl-1-oxopent-3-enyl)-4-prenylcyclopenta-1,3-diene-1,3,5-triol. In some embodiments, the method comprises converting the product into a tautomer, wherein the tautomer is 3,4-dihydroxy-2-(3-methyl-1-oxobutyl)-4-(4-methyl-1-oxopent-3-enyl)-5-prenylcyclopent-2-en-1-one.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentadiene of a product while the condensed phase is suspended in the gas phase, wherein: the reactant is 3,5,6-trihydroxy-2-(2-methyl-1-oxobutyl)-4,6-diprenylcyclohexa-2,4-dien-1-one; and the product is 2-(2-methyl-1-oxobutyl)-5-(4-methyl-1-oxopent-3-enyl)-4-prenylcyclopenta-1,3-diene-1,3,5-triol. In some embodiments, the method comprises converting the product into a tautomer, wherein the tautomer is 3,4-dihydroxy-2-(2-methyl-1-oxobutyl)-4-(4-methyl-1-oxopent-3-enyl)-5-prenylcyclopent-2-en-1-one.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentadiene of a product while the condensed phase is suspended in the gas phase, wherein: the reactant is 3,5,6-trihydroxy-2-(2-methyl-1-oxopropyl)-4,6-diprenylcyclohexa-2,4-dien-1-one; and the product is 2-(2-methyl-1-oxopropyl)-5-(4-methyl-1-oxopent-3-enyl)-4-prenylcyclopenta-1,3-diene-1,3,5-triol. In some embodiments, the method comprises converting the product into a tautomer, wherein the tautomer is 3,4-dihydroxy-2-(2-methyl-1-oxopropyl)-4-(4-methyl-1-oxopent-3-enyl)-5-prenylcyclopent-2-en-1-one.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentadiene of a product while the condensed phase is suspended in the gas phase, wherein: the reactant is 3,5,6-trihydroxy-2-(1-oxopropyl)-4,6-diprenylcyclohexa-2,4-dien-1-one; and the product is 2-(1-oxopropyl)-5-(4-methyl-1-oxopent-3-enyl)-4-prenylcyclopenta-1,3-diene-1,3,5-triol. In some embodiments, the method comprises converting the product into a tautomer, wherein the tautomer is 3,4-dihydroxy-2-(1-oxopropyl)-4-(4-methyl-1-oxopent-3-enyl)-5-prenylcyclopent-2-en-1-one.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentadiene of a product while the condensed phase is suspended in the gas phase, wherein: the reactant is 3,5,6-trihydroxy-2-(4-methyl-1-oxopentyl)-4,6-diprenylcyclohexa-2,4-dien-1-one; and the product is 2-(4-methyl-1-oxopentyl)-5-(4-methyl-1-oxopent-3-enyl)-4-prenylcyclopenta-1,3-diene-1,3,5-triol. In some embodiments, the method comprises converting the product into a tautomer, wherein the tautomer is 3,4-dihydroxy-2-(4-methyl-1-oxopentyl)-4-(4-methyl-1-oxopent-3-enyl)-5-prenylcyclopent-2-en-1-one.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentene of a product while the condensed phase is suspended in the gas phase, wherein: the cyclohexadiene comprises a first carbon atom, a second carbon atom, a third carbon atom, a fourth carbon atom, a fifth carbon atom, and a sixth carbon atom, wherein the first carbon atom is bound to the second carbon atom by a single bond, the second carbon atom is bound to the third carbon atom by a single bond, the third carbon atom is bound to the fourth carbon atom by a double bond, the fourth carbon atom is bound to the fifth carbon atom by a single bond, the fifth carbon atom is bound to the sixth carbon atom by a double bound, and the sixth carbon atom is bound to the first carbon atom by a single bond; the cyclopentene comprises the first carbon atom, the third carbon atom, the fourth carbon atom, the fifth carbon atom, and the sixth carbon atom, wherein the first carbon atom is bound to the third carbon atom by a single bond, the third carbon atom is bound to the fourth carbon atom by a single bond, the fourth carbon atom is bound to the fifth carbon atom by a single bond, the fifth carbon atom is bound to the sixth carbon atom by a single bound, and the sixth carbon atom is bound to the first carbon atom by a double bond; the product is a structural isomer of the reactant; the product comprises the second carbon atom, which is bound to the third carbon atom of the cyclopentene by a single bond; the first carbon atom of the cyclohexadiene is an unsaturated carbon atom that is substituted with an oxo, thioxo, or imino substituent, and the first carbon atom of the cyclopentene is an unsaturated carbon atom that is substituted with a hydroxy, sulfanyl, or amino substituent, respectively; the second carbon atom of the cyclohexadiene is a saturated carbon atom that is substituted with a hydroxy, sulfanyl, or amino substituent, and the second carbon atom of the product is an unsaturated carbon atom that is substituted with an oxo, thioxo, or imino substituent, respectively; the third carbon atom of the cyclohexadiene is an unsaturated carbon atom that is substituted with a hydroxy, sulfanyl, or amino substituent, and the third carbon atom of the cyclopentene is a saturated carbon atom that is substituted with a hydroxy, sulfanyl, or amino substituent, respectively; and the fifth carbon atom of the cyclohexadiene is an unsaturated carbon atom that is substituted with a hydroxy, sulfanyl, or amino substituent, and the fifth carbon atom of the product is an unsaturated carbon atom that is substituted with an oxo, thioxo, or imino substituent, respectively.

In some embodiments, the reactant has General Structure Ib; the product has General Structure IIb; either (i) R1a is oxo, and R1b is hydroxy, (ii) R1a is imino, and R1b is amino, or (iii) R1a is thioxo, and R1b is sulfanyl; either (i) R2a is hydroxy, and R2b is oxo, (ii) R2a is amino, and R2b is imino, or (iii) R2a is sulfanyl, and R2b is thioxo; R3 is either hydroxy, amino, or sulfanyl; R4, R6, and R7 are each independently selected from hydro, hydroxy, amino, cyano, halo, and a branched-or-unbranched, saturated-or-unsaturated, C1-C11 hydrocarbon group that is optionally substituted with one or more of hydroxy, oxo, sulfanyl, thioxo, thio, amino, cyano, isocyano, halo, phenyl, oxa, and a saturated-or-unsaturated cycloalkyl that is optionally substituted with one or more of hydroxy, methoxy, oxo, formyl, acetyl, methyl, ethyl, 2-propyl, and propen-2-yl; and either (i) R5a is hydroxy, and R5b is oxo, (ii) R5a is amino, and R5b is imino, or (iii) R5a is sulfanyl, and R5b is thioxo.

In some embodiments, R1a, R2b, and R5b are each oxo; and R1b, R2a, R3, and R5a are each hydroxy.

In some embodiments, R4 and R6 are each independently selected from hydro, hydroxy, methoxy, formyl, acetyl, 1-oxopropyl, 1-oxobutyl, 2-methyl-1-oxopropyl, 2-methyl-1-oxobutyl, 3-methyl-1-oxobutyl, 4-methyl-1-oxopentyl, methyl, ethyl, vinyl, ethynyl, propyl, prop-2-yl, prop-1-enyl, prop-2-enyl, propen-2-yl, prenyl, and geranyl; and R7 is methyl, ethyl, vinyl, propyl, prop-2-yl, prop-1-enyl, prop-2-enyl, propen-2-yl, prenyl, or geranyl.

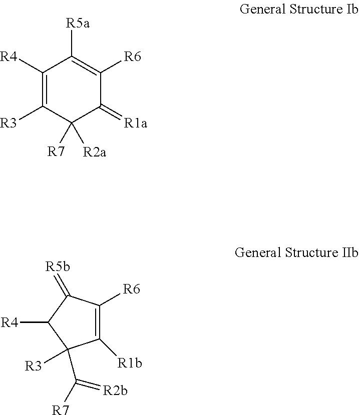

In some embodiments, R1a. R2b, and R5b are each oxo; Rib, R2a, R3, and R5a are each hydroxy; R4 and R7 are each prenyl; and R6 is 1-oxopropyl, 2-methyl-1-oxopropyl, 2-methyl-1-oxobutyl, 3-methyl-1-oxobutyl, or 4-methyl-1-oxopentyl.

In some embodiments, R6 is 1-oxopropyl.

In some embodiments, R6 is 2-methyl-1-oxopropyl.

In some embodiments, R6 is 2-methyl-1-oxobutyl.

In some embodiments, R6 is 3-methyl-1-oxobutyl.

In some embodiments, R6 is 4-methyl-1-oxopentyl.

In some embodiments, R4 of the product is not hydro; and converting the product into the reactant is stereoselective.

In some embodiments, the reactant is 3,5,6-trihydroxy-2-(3-methyl-1-oxobutyl)-4,6-diprenylcyclohexa-2,4-dien-1-one; and the product is 3,4-dihydroxy-2-(3-methyl-1-oxobutyl)-4-(4-methyl-1-oxopent-3-enyl)-5-prenylcyclopent-2-en-1-one.

In some embodiments, the reactant is 3,5,6-trihydroxy-2-(2-methyl-1-oxobutyl)-4,6-diprenylcyclohexa-2,4-dien-1-one; and the product is 3,4-dihydroxy-2-(2-methyl-1-oxobutyl)-4-(4-methyl-1-oxopent-3-enyl)-5-prenylcyclopent-2-en-1-one.

In some embodiments, the reactant is 3,5,6-trihydroxy-2-(2-methyl-1-oxopropyl)-4,6-diprenylcyclohexa-2,4-dien-1-one; and the product is 3,4-dihydroxy-2-(2-methyl-1-oxopropyl)-4-(4-methyl-1-oxopent-3-enyl)-5-prenylcyclopent-2-en-1-one.

In some embodiments, the reactant is 3,5,6-trihydroxy-2-(1-oxopropyl)-4,6-diprenylcyclohexa-2,4-dien-1-one; and the product is 3,4-dihydroxy-2-(1-oxopropyl)-4-(4-methyl-1-oxopent-3-enyl)-5-prenylcyclopent-2-en-1-one.

In some embodiments, the reactant is 3,5,6-trihydroxy-2-(4-methyl-1-oxopentyl)-4,6-diprenylcyclohexa-2,4-dien-1-one; and the product is 3,4-dihydroxy-2-(4-methyl-1-oxopentyl)-4-(4-methyl-1-oxopent-3-enyl)-5-prenylcyclopent-2-en-1-one.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentene of a product while the condensed phase is suspended in the gas phase, wherein: the reactant is 3,5,6-trihydroxy-2-(3-methyl-1-oxobutyl)-4,6-diprenylcyclohexa-2,4-dien-1-one; and the product is 3,4-dihydroxy-2-(3-methyl-1-oxobutyl)-4-(4-methyl-1-oxopent-3-enyl)-5-prenylcyclopent-2-en-1-one. In some embodiments, converting the cyclohexadiene of the reactant into the cyclopentene of the product comprises converting the reactant into 2-(3-methyl-1-oxobutyl)-5-(4-methyl-1-oxopent-3-enyl)-4-prenylcyclopenta-1,3-diene-1,3,5-triol and then tautomerizing the 2-(3-methyl-1-oxobutyl)-5-(4-methyl-1-oxopent-3-enyl)-4-prenylcyclopenta-1,3-diene-1,3,5-triol into the product.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentene of a product while the condensed phase is suspended in the gas phase, wherein: the reactant is 3,5,6-trihydroxy-2-(2-methyl-1-oxobutyl)-4,6-diprenylcyclohexa-2,4-dien-1-one; and the product is 3,4-dihydroxy-2-(2-methyl-1-oxobutyl)-4-(4-methyl-1-oxopent-3-enyl)-5-prenylcyclopent-2-en-1-one. In some embodiments, converting the cyclohexadiene of the reactant into the cyclopentene of the product comprises converting the reactant into 2-(2-methyl-1-oxobutyl)-5-(4-methyl-1-oxopent-3-enyl)-4-prenylcyclopenta-1,3-diene-1,3,5-triol and then tautomerizing the 2-(2-methyl-1-oxobutyl)-5-(4-methyl-1-oxopent-3-enyl)-4-prenylcyclopenta-1,3-diene-1,3,5-triol into the product.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentene of a product while the condensed phase is suspended in the gas phase, wherein: the reactant is 3,5,6-trihydroxy-2-(2-methyl-1-oxopropyl)-4,6-diprenylcyclohexa-2,4-dien-1-one; and the product is 3,4-dihydroxy-2-(2-methyl-1-oxopropyl)-4-(4-methyl-1-oxopent-3-enyl)-5-prenylcyclopent-2-en-1-one. In some embodiments, converting the cyclohexadiene of the reactant into the cyclopentene of the product comprises converting the reactant into 2-(2-methyl-1-oxopropyl)-5-(4-methyl-1-oxopent-3-enyl)-4-prenylcyclopenta-1,3-diene-1,3,5-triol and then tautomerizing the 2-(2-methyl-1-oxopropyl)-5-(4-methyl-1-oxopent-3-enyl)-4-prenylcyclopenta-1,3-diene-1,3,5-triol into the product.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentene of a product while the condensed phase is suspended in the gas phase, wherein: the reactant is 3,5,6-trihydroxy-2-(1-oxopropyl)-4,6-diprenylcyclohexa-2,4-dien-1-one; and the product is 3,4-dihydroxy-2-(1-oxopropyl)-4-(4-methyl-1-oxopent-3-enyl)-5-prenylcyclopent-2-en-1-one. In some embodiments, converting the cyclohexadiene of the reactant into the cyclopentene of the product comprises converting the reactant into 2-(1-oxopropyl)-5-(4-methyl-1-oxopent-3-enyl)-4-prenylcyclopenta-1,3-diene-1,3,5-triol and then tautomerizing the 2-(1-oxopropyl)-5-(4-methyl-1-oxopent-3-enyl)-4-prenylcyclopenta-1,3-diene-1,3,5-triol into the product.

Various aspects of this disclosure relate to a method to perform a ring contraction, comprising: (1) providing a condensed phase that comprises a reactant that comprises a cyclohexadiene; (2) suspending the condensed phase in a gas phase; and (3) transferring sufficient energy to the reactant to perform a ring contraction that converts the cyclohexadiene of the reactant into a cyclopentene of a product while the condensed phase is suspended in the gas phase, wherein: the reactant is 3,5,6-trihydroxy-2-(4-methyl-1-oxopentyl)-4,6-diprenylcyclohexa-2,4-dien-1-one; and the product is 3,4-dihydroxy-2-(4-methyl-1-oxopentyl)-4-(4-methyl-1-oxopent-3-enyl)-5-prenylcyclopent-2-en-1-one. In some embodiments, converting the cyclohexadiene of the reactant into the cyclopentene of the product comprises converting the reactant into 2-(4-methyl-1-oxopentyl)-5-(4-methyl-1-oxopent-3-enyl)-4-prenylcyclopenta-1,3-diene-1,3,5-triol and then tautomerizing the 2-(4-methyl-1-oxopentyl)-5-(4-methyl-1-oxopent-3-enyl)-4-prenylcyclopenta-1,3-diene-1,3,5-triol into the product.

In some embodiments, converting the cyclohexadiene of the reactant into the cyclopentene of the product creates two new stereocenters stereoselectively.

In some embodiments, converting the reactant into the product creates one new stereocenter stereoselectively.

In some embodiments, the ring contraction is stereoselective.

In some embodiments, both the second carbon atom of the reactant and the third carbon atom of the product have sinister (S) chirality.

In some embodiments, the substituent of the second carbon atom of the reactant comprises a proton; and the ring contraction comprises transferring the proton from the substituent of the second carbon atom of the reactant to the substituent of the first carbon atom of the reactant.

In some embodiments, the condensed phase comprises a solid phase. In some specific embodiments, the condensed phase comprises a solid phase that comprises the reactant.

In some embodiments, the condensed phase comprises a liquid phase.

In some embodiments, the condensed phase comprises a lipid phase. In some specific embodiments, the condensed phase comprises a lipid phase that comprises the reactant.

In some embodiments, the condensed phase lacks an aqueous phase that comprises the reactant.

In some embodiments, the condensed phase lacks a water-miscible phase that comprises the reactant.

In some embodiments, the condensed phase has a surface-area-to-volume ratio of at least 500 per meter. In some specific embodiments, the condensed phase has a surface-area-to-volume ratio of at least 1000 per meter. In some very specific embodiments, the condensed phase has a surface-area-to-volume ratio of at least 5000 per meter.

In some embodiments, providing the condensed phase comprises processing a starting material; the starting material has a surface-area-to-volume ratio; and the processing increases the surface-area-to-volume ratio. In some specific embodiments, processing the starting material comprises grinding the starting material. In some specific embodiments, the starting material has an average surface-area-to-volume ratio; and processing the starting material comprises selecting a portion of the starting material that has a greater surface-area-to-volume ratio than the average surface-area-to-volume ratio of the starting material.

In some embodiments, the condensed phase comprises a plant material. In some specific embodiments, the condensed phase comprises a plant material from Humulus lupulus.

In some embodiments, the ring contraction is a first-order chemical reaction.

In some embodiments, transferring sufficient energy to the reactant to perform the ring contraction comprises contacting the condensed phase with at least 0.0004 and no greater than 0.04 kilowatt hours of energy per gram of the condensed phase.

In some embodiments, transferring sufficient energy to the reactant to perform the ring contraction comprises contacting the condensed phase with energy at a rate of less than 100 kilowatts of power per gram of the condensed phase for a duration of less than 60 seconds

In some embodiments, transferring sufficient energy to the reactant to perform the ring contraction comprises contacting the condensed phase with a heated gas having a temperature of at least 105 and no greater than 250 degrees Celsius.

In some embodiments, transferring sufficient energy to the reactant to perform the ring contraction comprises contacting the condensed phase with a heated surface having a temperature of at least 105 and no greater than 250 degrees Celsius.

In some embodiments, transferring sufficient energy to the reactant to perform the ring contraction comprises heat transfer from the gas phase to the condensed phase. In some specific embodiments, transferring sufficient energy to the reactant to perform the ring contraction comprises sensible heat transfer from the gas phase to the condensed phase.

In some embodiments, transferring sufficient energy to the reactant to perform the ring contraction comprises heating the reactant to a temperature that is less than the boiling point of the reactant.

In some embodiments, transferring sufficient energy to the reactant to perform the ring contraction comprises heating the reactant to a temperature that is less than the boiling point of the product.

In some embodiments, transferring sufficient energy to the reactant to perform the ring contraction comprises irradiating the condensed phase. In some specific embodiments, transferring sufficient energy to the reactant to perform the ring contraction comprises irradiating the condensed phase with ultraviolet light. In some very specific embodiments, transferring sufficient energy to the reactant to perform the ring contraction comprises irradiating the condensed phase with ultraviolet A light.

In some embodiments, the method comprises vaporizing a molecule selected from either the reactant, the product, or a tautomer of the product. In some specific embodiments, the method comprises vaporizing a molecule selected from either the reactant, the product, or a tautomer of the product, wherein the molecule has a boiling point, and the vaporizing is performed at a temperature that is less than the boiling point of the molecule.

In some embodiments, the gas phase comprises a portion of the product; and the method comprises contacting the gas phase with a heat sink to condense the portion of the product into a distillate. In some specific embodiments, the gas phase comprises a portion of the product; the method comprises contacting the gas phase with a heat sink to condense the portion of the product into a distillate; the heat sink comprises a liquid; the distillate comprises the liquid; and the method comprises evaporating a majority of the liquid from the distillate to produce a finished product. In some very specific embodiments, the gas phase comprises a portion of the product; the method comprises contacting the gas phase with a heat sink to condense the portion of the product into a distillate; the heat sink comprises ethanol; the distillate comprises the ethanol; and the method comprises evaporating a majority of the ethanol from the distillate to produce a finished product.

In some embodiments, the method comprises separating the gas phase from the condensed phase, wherein the gas phase comprises a portion of the product; and condensing the portion of the product into a distillate. In some specific embodiments, the method comprises directing the gas phase and the condensed phase through a cyclone to separate the gas phase from the condensed phase. In some specific embodiments, the method comprises directing the gas phase through a filter to separate the gas phase from the condensed phase, wherein the filter is selected to inhibit the condensed phase from passing through the filter.

In some embodiments, the method comprises directing the condensed phase along a path having a length of at least 1 meter, wherein the condensed phase is contacted with the sufficient energy in the path. In some specific embodiments, the method comprises directing the condensed phase along a path having a length of at least 4 meters, wherein the condensed phase is contacted with the sufficient energy in the path. In some very specific embodiments, the method comprises directing the condensed phase along a path having a length of at least 8 meters, wherein the condensed phase is contacted with the sufficient energy in the path.

In some embodiments, the method comprises directing the condensed phase along the path at a velocity of at least 1 meter per second. In some specific embodiments, the method comprises directing the condensed phase along the path at a velocity of at least 2 meters per second. In some very specific embodiments, the method comprises directing the condensed phase along the path at a velocity of at least 5 meters per second.

In some embodiments, both (i) suspending the condensed phase in the gas phase and (ii) transferring sufficient energy to the reactant to perform the ring contraction are completed in less than 10 minutes. In some specific embodiments, both (i) suspending the condensed phase in the gas phase and (ii) transferring sufficient energy to the reactant to perform the ring contraction are completed in less than 2 minutes. In some very specific embodiments, both (i) suspending the condensed phase in the gas phase and (ii) transferring sufficient energy to the reactant to perform the ring contraction are completed in less than 30 seconds.

Various aspects of this disclosure relate to a composition produced according to a method described anywhere in this disclosure, wherein the composition comprises 2-acyl-5-(4-methyl-1-oxopent-3-enyl)-4-prenylcyclopenta-1,3-diene-1,3,5-triol; and acyl is selected from 1-oxopropyl, 2-methyl-1-oxopropyl, 2-methyl-1-oxobutyl, 3-methyl-1-oxobutyl, and 4-methyl-1-oxopentyl. In some embodiments, the composition comprises 2-acyl-3,4-dihydroxy-4-(4-methyl-1-oxopent-3-enyl)-5-prenylcyclopent-2-en-1-one.

Various aspects of this disclosure relate to a composition produced according to a method described anywhere in this disclosure, wherein the composition comprises 2-acyl-3,4-dihydroxy-4-(4-methyl-1-oxopent-3-enyl)-5-prenylcyclopent-2-en-1-one; and acyl is selected from 1-oxopropyl, 2-methyl-1-oxopropyl, 2-methyl-1-oxobutyl, 3-methyl-1-oxobutyl, and 4-methyl-1-oxopentyl. In some embodiments, the composition comprises 2-acyl-5-(4-methyl-1-oxopent-3-enyl)-4-prenylcyclopenta-1,3-diene-1,3,5-triol.

Various aspects of this disclosure relate to a composition, comprising 2-acyl-5-(4-methyl-1-oxopent-3-enyl)-4-prenylcyclopenta-1,3-diene-1,3,5-triol and cellulose; wherein acyl is selected from 1-oxopropyl, 2-methyl-1-oxopropyl, 2-methyl-1-oxobutyl, 3-methyl-1-oxobutyl, and 4-methyl-1-oxopentyl; and the composition lacks a protic polar solvent at a concentration greater than 20 percent by mass. In some embodiments, the composition comprises 2-acyl-3,4-dihydroxy-4-(4-methyl-1-oxopent-3-enyl)-5-prenylcyclopent-2-en-1-one.

Various aspects of this disclosure relate to a composition, comprising 2-acyl-3,4-dihydroxy-4-(4-methyl-1-oxopent-3-enyl)-5-prenylcyclopent-2-en-1-one and cellulose; wherein acyl is selected from 1-oxopropyl, 2-methyl-1-oxopropyl, 2-methyl-1-oxobutyl, 3-methyl-1-oxobutyl, and 4-methyl-1-oxopentyl; and the composition lacks a protic polar solvent at a concentration greater than 20 percent by mass. In some embodiments, the composition comprises 2-acyl-5-(4-methyl-1-oxopent-3-enyl)-4-prenylcyclopenta-1,3-diene-1,3,5-triol.

In some embodiments, the cellulose comprises cellulose I.

In some embodiments, the composition comprises protein that comprises amino acid sequences that encode a 2-acyl-4-prenylphloroglucinol 6-prenyltransferase.

“2-acyl-4-prenylphloroglucinol 6-prenyltransferase” refers to an enzyme capable of transferring a prenyl group to the 6-position of a 2-acyl-4-prenylphloroglucinol irrespective of the nature of the acyl of the 2-acyl-4-prenylphloroglucinol. Any limitation on the scope of “acyl” either in this disclosure or in any patent claim that matures from this document defines the scope of 2-acyl-4-prenylphloroglucinol 6-prenyltransferase to include 2-acyl-4-prenylphloroglucinol 6-prenyltransferase activity in relation to the full scope of “acyl” and does not limit the scope of the 2-acyl-4-prenylphloroglucinol 6-prenyltransferase activity to the scope of “acyl”. When “acyl” is 1-oxopropyl, for example, then a 2-acyl-4-prenylphloroglucinol 6-prenyltransferase has 2-(1-oxopropyl)-4-prenylphloroglucinol 6-prenyltransferase activity by definition, and the 2-acyl-4-prenylphloroglucinol 6-prenyltransferase may also have other 2-acyl-4-prenylphloroglucinol 6-prenyltransferase activity such as 2-(3-methyl-1-oxobutyl)-4-prenylphloroglucinol 6-prenyltransferase activity.

Various aspects of this disclosure relate to a composition, comprising 2-acyl-5-(4-methyl-1-oxopent-3-enyl)-4-prenylcyclopenta-1,3-diene-1,3,5-triol and protein that comprises amino acid sequences that encode a 2-acyl-4-prenylphloroglucinol 6-prenyltransferase; wherein acyl is selected from 1-oxopropyl, 2-methyl-1-oxopropyl, 2-methyl-1-oxobutyl, 3-methyl-1-oxobutyl, and 4-methyl-1-oxopentyl; and the composition lacks a protic polar solvent at a concentration greater than 20 percent by mass. In some embodiments, the composition comprises nucleic acid that comprises nucleotide sequences that encode the 2-acyl-4-prenylphloroglucinol 6-prenyltransferase. In some embodiments, the composition comprises 2-acyl-3,4-dihydroxy-4-(4-methyl-1-oxopent-3-enyl)-5-prenylcyclopent-2-en-1-one.

Various aspects of this disclosure relate to a composition, comprising 2-acyl-5-(4-methyl-1-oxopent-3-enyl)-4-prenylcyclopenta-1,3-diene-1,3,5-triol and nucleic acid that comprises nucleotide sequences that encode a 2-acyl-4-prenylphloroglucinol 6-prenyltransferase; wherein acyl is selected from 1-oxopropyl, 2-methyl-1-oxopropyl, 2-methyl-1-oxobutyl, 3-methyl-1-oxobutyl, and 4-methyl-1-oxopentyl; and the composition lacks a protic polar solvent at a concentration greater than 20 percent by mass. In some embodiments, the composition comprises protein that comprises amino acid sequences that encode the 2-acyl-4-prenylphloroglucinol 6-prenyltransferase. In some embodiments, the composition comprises 2-acyl-3,4-dihydroxy-4-(4-methyl-1-oxopent-3-enyl)-5-prenylcyclopent-2-en-1-one.

Various aspects of this disclosure relate to a composition, comprising 2-acyl-3,4-dihydroxy-4-(4-methyl-1-oxopent-3-enyl)-5-prenylcyclopent-2-en-1-one and protein that comprises amino acid sequences that encode a 2-acyl-4-prenylphloroglucinol 6-prenyltransferase; wherein acyl is selected from 1-oxopropyl, 2-methyl-1-oxopropyl, 2-methyl-1-oxobutyl, 3-methyl-1-oxobutyl, and 4-methyl-1-oxopentyl; and the composition lacks a protic polar solvent at a concentration greater than 20 percent by mass. In some embodiments, the composition comprises nucleic acid that comprises nucleotide sequences that encode the 2-acyl-4-prenylphloroglucinol 6-prenyltransferase. In some embodiments, the composition comprises 2-acyl-5-(4-methyl-1-oxopent-3-enyl)-4-prenylcyclopenta-1,3-diene-1,3,5-triol, Various aspects of this disclosure relate to a composition, comprising 2-acyl-3,4-dihydroxy-4-(4-methyl-1-oxopent-3-enyl)-5-prenylcyclopent-2-en-1-one and nucleic acid that comprises nucleotide sequences that encode a 2-acyl-4-prenylphloroglucinol 6-prenyltransferase; wherein acyl is selected from 1-oxopropyl, 2-methyl-1-oxopropyl, 2-methyl-1-oxobutyl, 3-methyl-1-oxobutyl, and 4-methyl-1-oxopentyl; and the composition lacks a protic polar solvent at a concentration greater than 20 percent by mass. In some embodiments, the composition comprises protein that comprises amino acid sequences that encode the 2-acyl-4-prenylphloroglucinol 6-prenyltransferase. In some embodiments, the composition comprises 2-acyl-5-(4-methyl-1-oxopent-3-enyl)-4-prenylcyclopenta-1,3-diene-1,3,5-triol.

In some embodiments, the composition comprises cellulose.

In some embodiments, the composition comprises cellulose I.

In some embodiments, the composition comprises protein that comprises amino acid sequences that encode a 2-acyl-4-prenylphloroglucinol 6-prenyltransferase.

In some embodiments, the composition comprises nucleic acid that comprises nucleotide sequences that encode a 2-acyl-4-prenylphloroglucinol 6-prenyltransferase.

In some embodiments, the composition has a surface-area-to-volume ratio of at least 500 per meter. In some specific embodiments, the composition has a surface-area-to-volume ratio of at least 1000 per meter. In some very specific embodiments, the composition has a surface-area-to-volume ratio of at least 5000 per meter.

In some embodiments, the composition is suspended in a gas phase.

In some embodiments, the composition is contained within a container. In some embodiments, the composition is contained within a container or a reactor.

In some embodiments, the composition comprises caryophyllene.

In some embodiments, the composition comprises caryophyllene and 4,12,12-trimethyl-9-methylene-5-oxatricyclo[8.2.0.0⁴⁶]dodecane. In some specific embodiments, the composition comprises caryophyllene and 4,12,12-trimethyl-9-methylene-5-oxatricyclo[8.2.0.0^{4,6}]dodecane at a ratio of at least 1:10 and no greater than 1,000:1 by mass.

In some embodiments, the composition comprises humulene.

In some embodiments, the composition comprises 1,5,9,9-tetramethyl-12-oxabicyclo[9.1.0]dodeca-4,7-diene. In some specific embodiments, the composition comprises humulene and 1,5,9,9-tetramethyl-12-oxabicyclo[9.1.0]dodeca-4,7-diene at a ratio of at least 1:1 and no greater than 10,000:1 by mass.

In some embodiments, the composition comprises 1,5,5,8-tetramethyl-12-oxabicyclo[9.1.0]dodeca-3,7-diene. In some specific embodiments, the composition comprises humulene and 1,5,5,8-tetramethyl-12-oxabicyclo[9.1.0]dodeca-3,7-diene at a ratio of at least 1:10 and no greater than 1,000:1 by mass.

In some embodiments, the composition comprises 3,7,10,10-tetramethyl-12-oxabicyclo[9.1.0]dodeca-3,7-diene. In some specific embodiments, the composition comprises humulene and 3,7,10,10-tetramethyl-12-oxabicyclo[9.1.0]dodeca-3,7-diene at a ratio of at least 1:1 and no greater than 10,000:1 by mass.

In some embodiments, the composition comprises 1,5,8,8-tetramethylbicyclo[8.1.0]undec-5-ene-2,9-diol. In some specific embodiments, the composition comprises humulene and 1,5,8,8-tetramethylbicyclo[8.1.0]undec-5-ene-2,9-diol at a ratio of at least 1:10 and no greater than 1,000:1 by mass.

In some embodiments, the composition comprises 4,8,11,11-tetramethyltricyclo[7.2.0.0^{2,4}]undecane-5,8-diol. In some specific embodiments, the composition comprises humulene and 4,8,11,11-tetramethyltricyclo[7.2.0.0^{2,4}]undecane-5,8-diol at a ratio of at least 1:10 and no greater than 1,000:1 by mass.

In some embodiments, the composition comprises 4,8,11,11-tetramethyltricyclo[6.3.0.0^{2,4}]undecane-5,9-diol. In some specific embodiments, the composition comprises humulene and 4,8,11,11-tetramethyltricyclo[6.3.0.0^{2,4}]undecane-5,9-diol at a ratio of at least 1:10 and no greater than 1,000:1 by mass.

In some embodiments, the composition comprises 6,6,9-trimethyl-2-methylene-4,8-cycloundecadien-1-ol. In some specific embodiments, the composition comprises humulene and 6,6,9-trimethyl-2-methylene-4,8-cycloundecadien-1-ol at a ratio of at least 1:1 and no greater than 10,000:1 by mass.

In some embodiments, the composition comprises water at a concentration of no greater than 20 percent by mass. In some specific embodiments, the composition comprises water at a concentration of no greater than 10 percent by mass.

In some embodiments, the composition comprises polar protic solvents at a concentration of no greater than 20 percent by mass. In some specific embodiments, the composition comprises polar protic solvents at a concentration of no greater than 10 percent by mass.

Various aspects of this disclosure relate to a reactor that contains a composition that comprises a condensed phase and a gas phase, wherein: the condensed phase is suspended in the gas phase; the gas phase comprises caryophyllene and humulene; the condensed phase comprises 2-acyl-3,5,6-trihydroxy-4,6-diprenylcyclohexa-2,4-dien-1-one and 2-acyl-5-(4-methyl-1-oxopent-3-enyl)-4-prenylcyclopenta-1,3-diene-1,3,5-triol; and acyl is selected from 1-oxopropyl, 2-methyl-1-oxopropyl, 2-methyl-1-oxobutyl, 3-methyl-1-oxobutyl, and 4-methyl-1-oxopentyl. In some embodiments, the condensed phase comprises 2-acyl-3,4-dihydroxy-(4-methyl-1-oxopent-3-enyl)-5-prenylcyclopent-2-en-1-one.

Various aspects of this disclosure relate to a reactor that contains a composition that comprises a condensed phase and a gas phase, wherein: the condensed phase is suspended in the gas phase; the gas phase comprises caryophyllene and humulene; the condensed phase comprises 2-acyl-3,5,6-trihydroxy-4,6-diprenylcyclohexa-2,4-dien-1-one and 2-acyl-3,4-dihydroxy-4-(4-methyl-1-oxopent-3-enyl)-5-prenylcyclopent-2-en-1-one; and acyl is selected from 1-oxopropyl, 2-methyl-1-oxopropyl, 2-methyl-1-oxobutyl, 3-methyl-1-oxobutyl, and 4-methyl-1-oxopentyl. In some embodiments, the condensed phase comprises 2-acyl-5-(4-methyl-1-oxopent-3-enyl)-4-prenylcyclopenta-1,3-diene-1,3,5-triol.

In some embodiments, the condensed phase comprises 3,5,6-trihydroxy-2-(3-methyl-1-oxobutyl)-4,6-diprenylcyclohexa-2,4-dien-1-one.

In some embodiments, the condensed phase comprises 2-(3-methyl-1-oxobutyl)-5-(4-methyl-1-oxopent-3-enyl)-4-prenylcyclopenta-1,3-diene-1,3,5-triol.

In some embodiments, the condensed phase comprises 3,4-dihydroxy-2-(3-methyl-1-oxobutyl)-4-(4-methyl-1-oxopent-3-enyl)-5-prenylcyclopent-2-en-1-one.

In some embodiments, the condensed phase comprises 3,5,6-trihydroxy-2-(2-methyl-1-oxobutyl)-4,6-diprenylcyclohexa-2,4-dien-1-one.

In some embodiments, the condensed phase comprises 2-(2-methyl-1-oxobutyl)-5-(4-methyl-1-oxopent-3-enyl)-4-prenylcyclopenta-1,3-diene-1,3,5-triol.

In some embodiments, the condensed phase comprises 3,4-dihydroxy-2-(2-methyl-1-oxobutyl)-4-(4-methyl-1-oxopent-3-enyl)-5-prenylcyclopent-2-en-1-one.

In some embodiments, the condensed phase comprises 3,5,6-trihydroxy-2-(2-methyl-1-oxopropyl)-4,6-diprenylcyclohexa-2,4-dien-1-one.

In some embodiments, the condensed phase comprises 2-(2-methyl-1-oxopropyl)-5-(4-methyl-1-oxopent-3-enyl)-4-prenylcyclopenta-1,3-diene-1,3,5-triol.

In some embodiments, the condensed phase comprises 3,4-dihydroxy-2-(2-methyl-1-oxopropyl)-4-(4-methyl-1-oxopent-3-enyl)-5-prenylcyclopent-2-en-1-one.

In some embodiments, the condensed phase comprises 3,5,6-trihydroxy-2-(1-oxopropyl)-4,6-diprenylcyclohexa-2,4-dien-1-one.

In some embodiments, the condensed phase comprises 2-(1-oxopropyl)-5-(4-methyl-1-oxopent-3-enyl)-4-prenylcyclopenta-1,3-diene-1,3,5-triol.

In some embodiments, the condensed phase comprises 3,4-dihydroxy-2-(1-oxopropyl)-4-(4-methyl-1-oxopent-3-enyl)-5-prenylcyclopent-2-en-1-one.

In some embodiments, the condensed phase comprises 3,5,6-trihydroxy-2-(4-methyl-1-oxopentyl)-4,6-diprenylcyclohexa-2,4-dien-1-one.

In some embodiments, the condensed phase comprises 2-(4-methyl-1-oxopentyl)-5-(4-methyl-1-oxopent-3-enyl)-4-prenylcyclopenta-1,3-diene-1,3,5-triol.

In some embodiments, the condensed phase comprises 3,4-dihydroxy-2-(4-methyl-1-oxopentyl)-4-(4-methyl-1-oxopent-3-enyl)-5-prenylcyclopent-2-en-1-one.

In some embodiments, the gas phase comprises 3,5,6-trihydroxy-2-(3-methyl-1-oxobutyl)-4,6-diprenylcyclohexa-2,4-dien-1-one.

In some embodiments, the gas phase comprises 2-(3-methyl-1-oxobutyl)-5-(4-methyl-1-oxopent-3-enyl)-4-prenylcyclopenta-1,3-diene-1,3,5-triol.

In some embodiments, the gas phase comprises 3,4-dihydroxy-2-(3-methyl-1-oxobutyl)-4-(4-methyl-1-oxopent-3-enyl)-5-prenylcyclopent-2-en-1-one.

In some embodiments, the gas phase comprises 3,5,6-trihydroxy-2-(2-methyl-1-oxobutyl)-4,6-diprenylcyclohexa-2,4-dien-1-one.

In some embodiments, the gas phase comprises 2-(2-methyl-1-oxobutyl)-5-(4-methyl-1-oxopent-3-enyl)-4-prenylcyclopenta-1,3-diene-1,3,5-triol.

In some embodiments, the gas phase comprises 3,4-dihydroxy-2-(2-methyl-1-oxobutyl)-4-(4-methyl-1-oxopent-3-enyl)-5-prenylcyclopent-2-en-1-one.

In some embodiments, the gas phase comprises 3,5,6-trihydroxy-2-(2-methyl-1-oxopropyl)-4,6-diprenylcyclohexa-2,4-dien-1-one.

In some embodiments, the gas phase comprises 2-(2-methyl-1-oxopropyl)-5-(4-methyl-1-oxopent-3-enyl)-4-prenylcyclopenta-1,3-diene-1,3,5-triol.

In some embodiments, the gas phase comprises 3,4-dihydroxy-2-(2-methyl-1-oxopropyl)-4-(4-methyl-1-oxopent-3-enyl)-5-prenylcyclopent-2-en-1-one.

In some embodiments, the gas phase comprises 3,5,6-trihydroxy-2-(1-oxopropyl)-4,6-diprenylcyclohexa-2,4-dien-1-one.

In some embodiments, the gas phase comprises 2-(1-oxopropyl)-5-(4-methyl-1-oxopent-3-enyl)-4-prenylcyclopenta-1,3-diene-1,3,5-triol.

In some embodiments, the gas phase comprises 3,4-dihydroxy-2-(1-oxopropyl)-4-(4-methyl-1-oxopent-3-enyl)-5-prenylcyclopent-2-en-1-one.

In some embodiments, the gas phase comprises 3,5,6-trihydroxy-2-(4-methyl-1-oxopentyl)-4,6-diprenylcyclohexa-2,4-dien-1-one.

In some embodiments, the gas phase comprises 2-(4-methyl-1-oxopentyl)-5-(4-methyl-1-oxopent-3-enyl)-4-prenylcyclopenta-1,3-diene-1,3,5-triol.

In some embodiments, the gas phase comprises 3,4-dihydroxy-2-(4-methyl-1-oxopentyl)-4-(4-methyl-1-oxopent-3-enyl)-5-prenylcyclopent-2-en-1-one.

In some embodiments, the composition comprises 1,5,9,9-tetramethyl-12-oxabicyclo[9.1.0]dodeca-4,7-diene. In some specific embodiments, the composition comprises humulene and 1,5,9,9-tetramethyl-12-oxabicyclo[9.1.0]dodeca-4,7-diene at a ratio of at least 1:1 and no greater than 10,000:1 by mass.

In some embodiments, the composition comprises 1,5,5,8-tetramethyl-12-oxabicyclo[9.1.0]dodeca-3,7-diene. In some specific embodiments, the composition comprises humulene and 1,5,5,8-tetramethyl-12-oxabicyclo[9.1.0]dodeca-3,7-diene at a ratio of at least 1:10 and no greater than 1,000:1 by mass.

In some embodiments, the composition comprises 3,7,10,10-tetramethyl-12-oxabicyclo[9.1.0]dodeca-3,7-diene. In some specific embodiments, the composition comprises humulene and 3,7,10,10-tetramethyl-12-oxabicyclo[9.1.0]dodeca-3,7-diene at a ratio of at least 1:1 and no greater than 10,000:1 by mass.

In some embodiments, the composition comprises 1,5,8,8-tetramethylbicyclo[8.1.0]undec-5-ene-2,9-diol. In some specific embodiments, the composition comprises humulene and 1,5,8,8-tetramethylbicyclo[8.1.0]undec-5-ene-2,9-diol at a ratio of at least 1:10 and no greater than 1,000:1 by mass.

In some embodiments, the composition comprises 4,8,11,11-tetramethyltricyclo[7.2.0.0^{2,4}]undecane-5,8-diol. In some specific embodiments, the composition comprises humulene and 4,8,11,11-tetramethyltricyclo[7.2.0.0^{2,4}]undecane-5,8-diol at a ratio of at least 1:10 and no greater than 1,000:1 by mass.

In some embodiments, the composition comprises 4,8,11,11-tetramethyltricyclo[6.3.0.0^{2,4}]undecane-5,9-diol. In some specific embodiments, the composition comprises humulene and 4,8,11,11-tetramethyltricyclo[6.3.0.0^{2,4}]undecane-5,9-diol at a ratio of at least 1:10 and no greater than 1,000:1 by mass.

In some embodiments, the composition comprises 6,6,9-trimethyl-2-methylene-4,8-cycloundecadien-1-ol. In some specific embodiments, the composition comprises humulene and 6,6,9-trimethyl-2-methylene-4,8-cycloundecadien-1-ol at a ratio of at least 1:1 and no greater than 10,000:1 by mass.

In some embodiments, the composition comprises 4,12,12-trimethyl-9-methylene-5-oxatricyclo[8.2.0.0^{4,6}]dodecane. In some specific embodiments, the composition comprises caryophyllene and 4,12,12-trimethyl-9-methylene-5-oxatricyclo[8.2.0.0^{4,6}]dodecane at a ratio of at least 1:10 and no greater than 1,000:1 by mass.

In some embodiments, the condensed phase comprises cellulose. In some specific embodiments, the condensed phase comprises cellulose I.

In some embodiments, the condensed phase comprises protein that comprises amino acid sequences that encode a 2-acyl-4-prenylphloroglucinol 6-prenyltransferase.

In some embodiments, the condensed phase comprises nucleic acid that comprises nucleotide sequences that encode a 2-acyl-4-prenylphloroglucinol 6-prenyltransferase.

In some embodiments, the condensed phase comprises plant material from Humulus lupulus.

In some embodiments, the condensed phase has a surface-area-to-volume ratio of at least 500 per meter. In some specific embodiments, the condensed phase has a surface-area-to-volume ratio of at least 1000 per meter. In some very specific embodiments, the condensed phase has a surface-area-to-volume ratio of at least 5000 per meter.

In some embodiments, the composition has an average temperature that is greater than 100 degrees Celsius. In some specific embodiments, the composition has an average temperature of at least 105 and no greater than 235 degrees Celsius.

### EXEMPLIFICATION

Humulus lupulus flowers are ground to a surface-area-to-volume ratio of greater than 5000 per meter. The ground flowers are suspended in a heated gas having a temperature of at least 105 and no greater than 235 degrees Celsius within a reactor. The heated gas and ground flowers are propelled along a path having a length of at least 4 meters at a velocity of at least 2 meters per second. The length of the path and the velocity are selected to contact the ground flowers with at least 0.0004 and no greater than 0.04 kilowatt hours of energy per gram of the ground flowers over a total time of less than 30 seconds. The heated gas and ground flowers are then separated using a cyclone and a filter. Volatile molecules that are distilled from the ground flowers are separated from the gas by condensation using a heat sink to produce a distillate. The heat sink comprises ethanol, which reduces the viscosity of the distillate and improves pumping. A majority of the ethanol is removed from the distillate by evaporation to produce a finished product.

Two compositions are collected from the reactor: (1) the minimally-processed ground hops flowers and (2) the distillate. Both the minimally-processed ground hops flowers and the distillate comprise the cyclopentadienes 2-(3-methyl-1-oxobutyl)-5-(4-methyl-1-oxopent-3-enyl)-4-prenylcyclopenta-1,3-diene-1,3,5-triol; 2-(2-methyl-1-oxobutyl)-5-(4-methyl-1-oxopent-3-enyl)-4-prenylcyclopenta-1,3-diene-1,3,5-triol; 2-(2-methyl-1-oxopropyl)-5-(4-methyl-1-oxopent-3-enyl)-4-prenylcyclopenta-1,3-diene-1,3,5-triol; 2-(1-oxopropyl)-5-(4-methyl-1-oxopent-3-enyl)-4-prenylcyclopenta-1,3-diene-1,3,5-triol; and 2-(4-methyl-1-oxopentyl)-5-(4-methyl-1-oxopent-3-enyl)-4-prenylcyclopenta-1,3-diene-1,3,5-triol. The cyclopentadienes tautomerize into the cyclopentenes 3,4-dihydroxy-2-(3-methyl-1-oxobutyl)-4-(4-methyl-1-oxopent-3-enyl)-5-prenylcyclopent-2-en-1-one; 3,4-dihydroxy-2-(2-methyl-1-oxobutyl)-4-(4-methyl-1-oxopent-3-enyl)-5-prenylcyclopent-2-en-1-one; 3,4-dihydroxy-2-(2-methyl-1-oxopropyl)-4-(4-methyl-1-oxopent-3-enyl)-5-prenylcyclopent-2-en-1-one; 3,4-dihydroxy-2-(1-oxopropyl)-4-(4-methyl-1-oxopent-3-enyl)-5-prenylcyclopent-2-en-1-one; and 3,4-dihydroxy-2-(4-methyl-1-oxopentyl)-4-(4-methyl-1-oxopent-3-enyl)-5-prenylcyclopent-2-en-1-one. Tautomerization proceeds slowly in the ground flowers, in which the cyclopentadienes are not solvated by a polar protic solvent, relative to in the condensed volatile molecules, in which the cyclopentadienes are exposed to the polar protic solvent ethanol.

1. A method to perform a ring contraction, comprising:
providing a condensed phase that comprises a reactant that comprises a cyclohexadiene;
suspending the condensed phase in a gas phase; and
transferring sufficient energy to the reactant to perform a ring contraction that converts the cyclohexadiene of the reactant into a cyclopentadiene of a product while the condensed phase is suspended in the gas phase,
wherein:
the cyclohexadiene comprises a first carbon atom, a second carbon atom, a third carbon atom, a fourth carbon atom, a fifth carbon atom, and a sixth carbon atom, wherein the first carbon atom is bound to the second carbon atom by a single bond, the second carbon atom is bound to the third carbon atom by a single bond, the third carbon atom is bound to the fourth carbon atom by a double bond, the fourth carbon atom is bound to the fifth carbon atom by a single bond, the fifth carbon atom is bound to the sixth carbon atom by a double bound, and the sixth carbon atom is bound to the first carbon atom by a single bond;
the cyclopentadiene comprises the first carbon atom, the third carbon atom, the fourth carbon atom, the fifth carbon atom, and the sixth carbon atom, wherein the first carbon atom is bound to the third carbon atom by a single bond, the third carbon atom is bound to the fourth carbon atom by a single bond, the fourth carbon atom is bound to the fifth carbon atom by a double bond, the fifth carbon atom is bound to the sixth carbon atom by a single bound, and the sixth carbon atom is bound to the first carbon atom by a double bond;
the product is a structural isomer of the reactant;
the product comprises the second carbon atom, which is bound to the third carbon atom of the cyclopentadiene by a single bond;
the first carbon atom of the cyclohexadiene is an unsaturated carbon atom that is substituted with an oxo, thioxo, or imino substituent, and the first carbon atom of the cyclopentadiene is an unsaturated carbon atom that is substituted with a hydroxy, sulfanyl, or amino substituent, respectively;
the second carbon atom of the cyclohexadiene is a saturated carbon atom that is substituted with a hydroxy, sulfanyl, or amino substituent, and the second carbon atom of the product is an unsaturated carbon atom that is substituted with an oxo, thioxo, or imino substituent, respectively; and
the third carbon atom of the cyclohexadiene is an unsaturated carbon atom that is substituted with a hydroxy, sulfanyl, or amino substituent, and the third carbon atom of the cyclopentadiene is a saturated carbon atom that is substituted with a hydroxy, sulfanyl, or amino substituent, respectively.

2. The method of claim 1, wherein:
the reactant has General Structure Ia;
the product has General Structure IIa;
either R1a is oxo, and R1b is hydroxy; R1a is imino, and R1b is amino; or R1a is thioxo, and R1b is sulfanyl;
either R2a is hydroxy, and R2b is oxo; R2a is amino, and R2b is imino; or R2a is sulfanyl, and R2b is thioxo;
R3 is either hydroxy, amino, or sulfanyl;
R4, R5, R6, and R7 are each independently selected from hydro, hydroxy, amino, cyano, halo, and a branched-or-unbranched, saturated-or-unsaturated, C1-C11 hydrocarbon group that is optionally substituted with one or more of hydroxy, oxo, sulfanyl, thioxo, thio, amino, cyano, isocyano, halo, phenyl, oxa, and a saturated-or-unsaturated cycloalkyl that is optionally substituted with one or more of hydroxy, methoxy, oxo, formyl, acetyl, methyl, ethyl, 2-propyl, and propen-2-yl; and
the dotted line in General Structure IIa depicts a double bond.

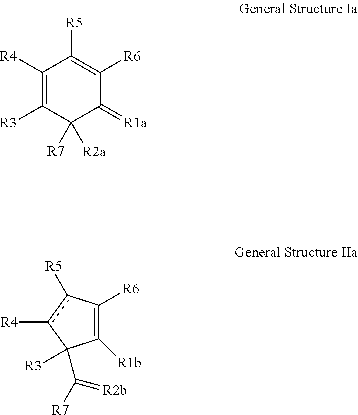

3. The method of claim 2, wherein R1a and R2b are each oxo; and R1b, R2a, and R3 are each hydroxy.

4-213. (canceled)
